(19) United States
(12) Patent Application Publication
(10) Pub. No.: US 2009/0247493 A1
(43) Pub. Date: Oct. 1, 2009

(54) Regimens for Treatment of Conditions Related to Estrogen Deficiency

(75) Inventors: Charles E. Diliberti, Montclair, NJ (US); Kathleen Z. Reape, Newtown Square, PA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Duramed Pharmaceuticals, Inc., Cincinnati, OH (US)

(21) Appl. No.: 12/162,445

(22) PCT Filed: Jan. 29, 2007
(86) PCT No.: PCT/US07/02455

§ 371 Date: Dec. 15, 2008

Related U.S. Application Data
(60) Provisional application No. 60/762,497, filed on Jan. 27, 2006.

(51) Int. Cl. A61K 31/56

(52) U.S. Cl. 514/170

(57) ABSTRACT

The present invention relates to methods of stimulating estrogen production that can be used to treat peri-menopausal or estrogen deficiency conditions utilizing regimens involving administration of estrogen and progestin, followed by a hormone-free period.

### BACKGROUND OF THE INVENTION

1. Field of the Invention

The present invention relates to methods of stimulating estrogen production that can be used to treat peri-menopausal or estrogen deficiency conditions utilizing regimens involving administration of estrogen and progestin, followed by a hormone-free period.

2. Related Art

Estrogen replacement therapy relates to treatment of disorders and conditions associated with menopause, peri-menopause, amenorrhea, and estrogen deficiency conditions. Menopause typically occurs in women during middle age and is often described as an ovarian shutdown. Menopause is usually associated with a profound decrease in circulating levels of estrogens. A peri-menopausal female can be described as a woman who has not yet definitely arrived at menopause but who is experiencing symptoms associated with menopause. It encompasses the years preceding the last menstrual period during which ovarian function declines and ultimately ceases and can include the presence of symptoms and irregular cycles. Amenorrhea can result from anovulation due to hormonal abnormalities, such as: decreased secretion of estrogen, gonadotropins, luteinizing hormone, and follicle stimulating hormone (FSH); a lack of ovarian response to gonadotropins; or constant presence of progesterone or other endocrine abnormalities. Currently, there are a large variety of disorders and conditions that are attributed to the reduction of estrogen levels. These disorders and conditions include hot flashes, dryness and atrophy of the vagina, parathesia, dyspareunia, osteoporosis, and an increase in cardiovascular disease. In an effort to reduce these disorders and conditions, estrogens are administered to women in a so-called “estrogen replacement therapy.” Estrogen replacement therapy continues to be the primary treatment of such disorders and conditions associated with menopause.

One of the risks associated with the administration of estrogen replacement therapy is that women with intact uteri may develop endometrial hyperplasia. The term “endometrial hyperplasia” refers to the over stimulation of the lining of the uterus, which is a precursor to endometrial or uterine cancer. The development of endometrial hyperplasia is a significant issue with estrogen replacement therapy. For example, it has been observed in U.S. Pat. No. RE 36,247 to Plunkett et al., and U.S. Pat. No. 5,043,331 to Hirvonen, that the co-administration of progestin can blunt the effect of estrogens. However, side effects often still occur with this co-administration. Thus, there continues to be a need for methods of stimulating estrogen production.

### BRIEF SUMMARY OF THE INVENTION

The present invention is directed to a method of stimulating estrogen production in a pre-menopausal or peri-menopausal female in need thereof, the method comprising administering to the female a combination of estrogen and progestin for a period of 21 or more consecutive days, immediately followed by a hormone-free period of 11 or more consecutive days.

The present invention is also directed to a method of stimulating estrogen production in a pre-menopausal or peri-menopausal female in need thereof the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

The present invention is also directed to a method of treating estrogen deficiency in a female in need thereof, the method comprising administering to the female a combination of estrogen and progestin for a period of 21 or more consecutive days, immediately followed by a hormone-free period of 11 or more consecutive days.

The present invention is also directed to a method of treating estrogen deficiency in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

The present invention is also directed to a method of restoring estrogen to a pre-menopausal level in a female in need thereof, the method comprising administering to the female a combination of estrogen and progestin for a period of 21 or more consecutive days, immediately followed by a hormone-free period of 11 or more consecutive days.

The present invention is also directed to a method of restoring estrogen to a pre-menopausal level in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

The present invention is also directed to a method of treating amenorrhea in a female in need thereof, the method comprising administering to the female a combination of estrogen and progestin for a period of 21 or more consecutive days, immediately followed by a hormone-free period of 11 or more consecutive days.

The present invention is also directed to a method of treating amenorrhea in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

The present invention is also directed to a method of treating anovulation in a female in need thereof, the method comprising administering to the female a combination of estrogen and progestin for a period of 21 or more consecutive days, immediately followed by a hormone-free period of 11 or more consecutive days.

The present invention is also directed to a method of treating anovulation in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

The present invention is also directed to a method of treating or preventing a disease, a disorder, or a symptom associated with deficient endogenous levels of estrogen in a female in need thereof, the method comprising administering to the female a combination of estrogen and progestin for a period of 21 or more consecutive days, immediately followed by a hormone-free period of 11 or more consecutive days.

The present invention is also directed to a method of treating or preventing a disease, a disorder, or a symptom associated with deficient endogenous levels of estrogen in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

The present invention is also directed to a method of increasing fertility in a female in need thereof, the method comprising administering to the female a combination of estrogen and progestin for a period of 21 or more consecutive days, immediately followed by a hormone-free period of 11 or more consecutive days.

The present invention is also directed to a method of increasing fertility in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

The present invention is also directed to a pharmaceutical kit comprising 21 or more daily doses of estrogen, 21 or more daily doses of progestin, 11 or more daily doses of a placebo pill, and a label comprising a direction for administering the placebo pill after the estrogen and progestin.

In some embodiments, the combination of estrogen and progestin is administered for a period of 21 to 119 consecutive days or a period of 21 to 91 consecutive days.

In some embodiments, the hormone-free period is 11 consecutive days to 91 consecutive days.

In some embodiments, the estrogen that is administered in combination with the progestin for a period of 21 or more consecutive days is administered in a daily amount equivalent to about 10 μg to about 50 μg of ethinyl estradiol, and the progestin that is administered in combination with the estrogen for a period of 21 or more consecutive days is administered in a daily amount equivalent to about 0.05 mg to about 0.5 mg of levonorgestrel.

In other embodiments, the estrogen that is administered in combination with the progestin is administered by an oral tablet. In other embodiments, the progestin that is administered in combination with the estrogen is administered by an oral tablet. In some embodiments, the combination of both estrogen and progestin that is administered is administered by an oral tablet.

In other embodiments, the hormone-free period is followed by a combination of estrogen and progestin for a period of 21 or more consecutive days, immediately followed by a hormone-free period of 11 or more consecutive days.

In some embodiments, the estrogen without progestin that is administered for a period of 1 to 14 consecutive days is administered in a daily amount equivalent to about 5 μg to about 50 μg of ethinyl estradiol.

In some embodiments, the hormone-free period is followed by a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

In some embodiments, the female has free estradiol levels of about 40 to about 150 pg/ml during early follicular phase of a female's menstrual cycle following the administration of estrogen and progestin and during the hormone-free period.

In some embodiments, the female has free estradiol levels at about 40 to about 350 pg/ml during late follicular phase of a female's menstrual cycle following the administration of estrogen and progestin and during the hormone-free period.

In other embodiments, the female has free estradiol levels at about 150 to about 750 pg/ml during luteal phase of a female's menstrual cycle following the administration of estrogen and progestin and during the hormone-free period.

### BRIEF DESCRIPTION OF THE FIGURES

FIG. 1 shows the endogenous hormone levels in a female subject given 84 consecutive daily doses of levonorgestrel/ethinyl estradiol tablet (150 μg/30 μg) followed by 7 consecutive daily doses of a 30 μg ethinyl estradiol tablet. Blood concentrations of endogenous hormones (follicle stimulating hormone (FSH) (solid square), luteinizing hormone (LH) (solid diamond), estradiol (hollow triangle), total testosterone (x), and free testosterone (star) were measured at various times during treatment and after completion of treatment.

### DETAILED DESCRIPTION OF THE INVENTION

The present invention provides an estrogen/progestin regimen or an estrogen-bridged estrogen/progestin regimen followed by a hormone-free period that is useful for stimulating estrogen production in peri-menopausal female and in the treatment of estrogen deficiency conditions in females. Ben-Maimon et al. WO 2004/098517, relates to the administration of extended cycle estrogen/progestin regimens to provide non-contraceptive benefits. Bell et al., U.S. Appl. Publ. No. 2003/0139381 A1, and Bell et al., U.S. Appl. Publ. No. 2005/0143359 A1, relate to the administration of estrogen-bridged estrogen/progestin regimens to provide contraceptive benefits and non-contraceptive benefits, respectively. WO 2004/098517, U.S. Appl. Publ. No. 2003/0139381 A1, and U.S. Appl. Publ. No. 2005/0143359 A1 are each fully incorporated by reference herein in their entirety.

### Regimens

In the methods of the present invention, a female with peri-menopausal levels of estrogen is administered an estrogen/progestin regimen or an estrogen-bridged estrogen/progestin regimen followed by a hormone-free period.

In the estrogen/progestin regimen, estrogen and progestin are administered to a female for a period of 21 or more consecutive days, immediately followed by a hormone-free period of 11 or more consecutive days where neither estrogen or progestin are administered. In some embodiments, estrogen and progestin are administered to a subject for a period of 21 to 27 consecutive days, immediately followed by a hormone-free period of 11 or more consecutive days where neither estrogen or progestin are administered.

In other embodiments of the estrogen/progestin regimen, the estrogen and progestin are administered for a period of 21 to 26 consecutive days, 23 to 25 consecutive days, or 25 to 26 consecutive days. In some embodiments, estrogen and progestin are administered for a period of 25 consecutive days. In some embodiments, the period of administration of estrogen and progestin is 21 to 119 consecutive days or 21 to 91 consecutive days.

In some embodiments, the period of administration of estrogen and progestin is 21 to 84 consecutive days, 21 to 77 consecutive days, 21 to 70 consecutive days, or 21 to 63 consecutive days. In other embodiments, the period of administration of estrogen and progestin is 28 to 84 consecutive days, 35 to 77 consecutive days, 42 to 70 consecutive days, or 49 to 63 consecutive days. In some embodiments, the period of administration of estrogen and progestin is 56 consecutive days.

The period of administration of estrogen and progestin is immediately followed by a period of 11 or more consecutive days during which neither estrogen nor progestin is administered (“hormone-free period”). In some embodiments of the invention, the hormone-free period is 11 to 91 consecutive days, 14 to 84 consecutive days, 14 to 77 consecutive days, 14 to 70 consecutive days, 14 to 63 consecutive days, 14 to 56 consecutive days, 14 to 49 consecutive days, 14 to 42 consecutive days, 14 to 35 consecutive days, or 14 to 28 consecutive days. In other embodiments of the invention, the hormone-free period is 21 to 77 consecutive days, 28 to 70 consecutive days, or 49 to 56 consecutive days.

In some embodiments, hormone-free placebo pills are administered during the hormone-free period.

In other aspects of the present invention, the female is administered an estrogen-bridged estrogen/progestin regimen with a hormone-free period. The terms “estrogen-bridged estrogen/progestin regimen” or “bridged regimen” refers to a regimen in which estrogen and progestin are administered to a subject for a period of 21 or more consecutive days, immediately followed by administration of estrogen (without progestin) for a period of 1 to 14 consecutive days (“unopposed estrogen period”), and then immediately followed by a hormone-free period of 11 to 91 consecutive days.

In some embodiments of the bridged regimen, the estrogen and progestin are administered for a period of 21 to 26 consecutive days, 23 to 25 consecutive days, or 25 to 26 consecutive days. In some embodiments, estrogen and progestin are administered for a period of 25 consecutive days.

In some embodiments, the estrogen and progestin are administered for a period of 21 to 119 consecutive days or 21 to 91 consecutive days. In some embodiments, the period of administration of estrogen and progestin is 21 to 84 consecutive days, 21 to 77 consecutive days, 21 to 70 consecutive days, or 21 to 63 consecutive days. In other embodiments, the period of administration of estrogen and progestin is 28 to 84 consecutive days, 35 to 77 consecutive days, 42 to 70 consecutive days, or 49 to 63 consecutive days. In some embodiments, the period of administration of estrogen and progestin is 56 consecutive days.

In some embodiments, the unopposed estrogen period is 1 to 14 consecutive days, 1 to 10 consecutive days, 2 to 8 consecutive days, 3 to 8 consecutive days, 1 to 7 consecutive days, 2 to 7 consecutive days, or 3 to 7 consecutive days. In yet other embodiments, the unopposed estrogen period is 2 to 5 consecutive days, 3 to 5 consecutive days, or 2 to 3 consecutive days. In some embodiments, the unopposed estrogen period is 3, 5, or 7 consecutive days.

The period of administration of estrogen and progestin is immediately followed by a period of 11 or more consecutive days during which neither estrogen nor progestin is administered (“hormone-free period”). In some embodiments of the invention, the hormone-free period is 11 to 91 consecutive days, 14 to 84 consecutive days, 14 to 77 consecutive days, 14 to 70 consecutive days, 14 to 63 consecutive days, 14 to 56 consecutive days, 14 to 49 consecutive days, 14 to 42 consecutive days, 14 to 35 consecutive days, or 14 to 28 consecutive days. In other embodiments of the invention, the hormone-free period is 21 to 77 consecutive days, 28 to 70 consecutive days, or 49 to 56 consecutive days.

In some embodiments, hormone-free placebo pills are administered during the hormone-free period.

The bridged regimen is optionally administered with an antidepressant. In some aspects of the invention, the antidepressant is administered in combination with estrogen during the unopposed estrogen interval of the bridged regimen. In other aspects of the invention, the antidepressant is administered continuously throughout the regimens, or, in yet other aspects of the invention, the antidepressant is administered intermittently. In yet other aspects of the invention, the antidepressant is administered one time during one cycle of the regimens of the present invention.

In a regimen of the present invention, the estrogen and progestin can be administered monophasically, biphasically, triphasically, or multiphasically. As used herein, “monophasic” refers to the continuous use of one particular dose of estrogen and progestin during the period of administration of estrogen and progestin. “Biphasic” refers to administration of a first continuous dose of estrogen and progestin during a first portion of the period of administration of the estrogen and progestin, with administration of a second continuous dose of estrogen and progestin during the second portion of the period of administration of the estrogen and progestin. “Triphasic” refers to administration of first, second, and third continuous doses of estrogen and progestin during the first, second, and third portions, respectively, of the period of administration of the estrogen and progestin. “Multiphasic” refers to administration of four or more continuous doses of estrogen and progestin during the first, second, third, and fourth or more portions, respectively, of the period of administration of the estrogen and progestin.

The regimens of the present invention can include administration to a female beginning at Day 1 of a menstrual cycle or medically induced withdrawal bleeding episode that is defined as beginning at the first day of menstrual flow. In alternative embodiments, the regimens of the present invention can also include administration to the female beginning at Day 1 of a menstrual cycle that is defined as beginning at the day after the ending of the menstrual flow. In alternative embodiments, the regimens of the present invention also can include administration to the female beginning at Day 1 of a menstrual cycle that is defined as beginning at any day within the menstrual cycle.

### Methods of Treatment

The regimens of the present invention disclosed herein are useful in the treatment or prevention conditions related to hypoestrogenism, i.e., low serum estrogen levels, in females. The regimens of the present invention can be administered to a peri-menopausal female. The regimens of the present invention can be administered to females, including pre-menopausal females, who exhibit conditions such as anovulation, amenorrhea, secondary amenorrhea or diseases, disorders, or symptoms associated with deficient levels of endogenous levels of estrogen. The regimens of the present invention can be administered to females to increase fertility.

The present invention is directed to a method of stimulating estrogen production in a peri-menopausal female in need thereof by administering to the female a regimen of the present invention, disclosed herein.

The term “stimulating estrogen production” as used herein, means stimulating the ovary to produce estrogen compared to endogenous levels or pretreatment levels of estrogen.

As used herein, “female” refers to any animal classified as a mammal, including humans and non-humans, such as, but not limited to, domestic and farm animals, zoo animals, sports animals, and pets.

“Peri-menopausal female” refers to a woman who has not yet definitely arrived at menopause but who is experiencing symptoms associated with menopause. “Peri-menopause” means “about or around the time of menopause.” It encompasses the years preceding the last menstrual period during which ovarian function declines and ultimately ceases and can include the presence of symptoms and irregular cycles. Menopause or post-menopause is the permanent cessation of menstruation after the loss of ovarian activity and is generally defined clinically as the absence of menstruation for about one year. Menopause may occur naturally in a woman or it may be artificially induced, e.g., through surgical or chemical means.

The terms “treat” and “treatment” as used herein refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the condition, disorder or disease progression; amelioration of the condition, disorder or disease state, remission (whether partial or total) the condition, disorder or disease, whether detectable or undetectable; or enhancement or improvement of the condition, disorder or disease. Treatment includes eliciting a clinically significant response, without excessive levels of side effects. Treatment also includes prolonging survival as compared to expected survival if not receiving treatment.

The term “continuous” or “consecutive,” as used herein in reference to “administration,” means that the frequency of administration is at least once daily. Note, however, that the frequency of administration can be greater than once daily and still be “continuous,” e.g., twice or even three times daily, as long as the dosage levels as specified herein are not exceeded.

The terms “dosage” and “dosage level,” as used herein, mean the total amount of estrogen or progestin administered per day. Thus, for example, “continuous administration” of a estrogen to a woman at a “dosage level” of 30 μg means that the woman receives a total of 30 μg of estrogen on a daily basis, whether the estrogen is administered as a single 30 μg dose or, e.g., three separate 10 μg doses. A conventional means of continuously administering an estrogen or progestin is as a single daily oral dose at the prescribed dosage level.

In some aspects of the invention, the disclosed methods are particularly useful in peri-menopausal females. Peri-menopausal women frequently experience a large variety of conditions and disorders that have been attributed to estrogen deprivation due to ovarian failure or hypoestrogenism. The duration of these disorders can be extremely variable and include hot flushes which can be devastating in some women and very mild in others. Dryness of the vagina associated with susceptibility to minor infections, and frequently associated with discomfort during intercourse, is another symptom that can be directly related to the decrease in estrogen availability.

In a long-term sense, one of the most health-threatening aspects of menopause is the loss of mineral from bone which can result in a decrease in bone mass (osteoporosis) and generates a serious risk of fractures. For example, evidence exists that there is a six-fold increase in fractures in post-menopausal women as opposed to men of the same age (Garraway et al., Mayo Clinic Proceedings 54: 701-707 (1979)). These fractures, of course, carry a high complication rate among older people, a marked increase in disability and general morbidity, and certainly an increased risk of mortality. Accordingly, the invention is directed to a method for treating conditions, such as the physical conditions described above, resulting from menopausal estrogen levels in a pre-menopausal or peri-menopausal, by administering a regimen of the present invention, disclosed herein, to the female. The invention is also directed to a method for treating conditions, such as the physical conditions described above, resulting from hypoestrogenism in a female by administering a regimen of the present invention, disclosed herein to the female. The invention is further directed to a method for treating conditions, such as the physical conditions described above, resulting from ovarian failure in a female by administering a regimen of the present invention, disclosed herein to the female.

The present invention is also directed to a method of treating estrogen deficiency in a female in need thereof by administering to the female a regimen of the present invention, disclosed herein.

Estrogen deficiency can occur for a variety of reasons. The present invention is directed to treating deficient levels of estrogen, regardless of the cause. Causes anticipated by the present invention are, but not limited to, natural menopause, peri-menopause, post-menopause, hypogonadism, or primary ovarian failure.

The present invention is also directed to a method of restoring estrogen to a pre-menopausal level in a female in need thereof by administering to the female a regimen of the present invention, disclosed herein.

The menstrual cycle in a normally menstruating female of child bearing age can be broken down into several phases, including, the early follicular phase, late follicular phase, midcycle peak, and luteal phase. In a normally menstruating female of child bearing age, these phases have different ranges of free estradiol levels that can vary based on the assay used to measure such free estradiol levels. In one example, a radioimmunoassay developed by Quest Diagnostics Nichols Institute (San Juan Capistrano, Calif.) can be used to measure free estradiol levels in a female. An assay performed by the Quest Diagnostics Nichols Institute recorded free estradiol levels in each phase for a female of child bearing age in the following ranges: early follicular phase (20-150 pg/ml), late follicular phase (40-350 pg/ml), midcycle peak (150-750 pg/ml), luteal phase (30-450 pg/ml). Free estradiol levels in a postmenopausal female measured by the same assay were 20 pg/ml or less.

In some embodiments, the free estradiol levels of a peri-menopausal female or a female with an estrogen deficiency condition are about 40 pg/ml to about 150 pg/ml in early follicular phase after administering the female a regimen of the present invention. In other embodiments, the free estradiol levels of a peri-menopausal female or a female with an estrogen deficiency condition are about 50 pg/ml to about 130 pg/ml, about 50 pg/ml to about 110 pg/ml, about 50 pg/ml to about 90 pg/ml or about 50 pg/ml to about 70 pg/ml in early follicular phase after administering the female a regimen of the present invention. In some embodiments, the free estradiol levels of a peri-menopausal female or a female with an estrogen deficiency condition are about 50 pg/ml in early follicular phase after administering the female a regimen of the present invention.

In some embodiments, the free estradiol levels of a peri-menopausal female or a female with an estrogen deficiency condition are about 40 pg/ml to about 350 pg/ml in late follicular phase after administering the female a regimen of the present invention. In other embodiments, the free estradiol levels of a peri-menopausal female or a female with an estrogen deficiency condition are about 50 pg/ml to about 300 pg/ml, about 50 pg/ml to about 250 pg/ml, about 50 pg/ml to about 200 pg/ml, about 50 pg/ml to about 150 pg/ml, about 50 pg/ml to about 130 pg/ml, about 50 pg/ml to about 110 pg/ml, about 50 pg/ml to about 90 pg/ml or about 50 pg/ml to about 70 pg/ml in late follicular phase after administering the female a regimen of the present invention. In some embodiments, the free estradiol levels of a peri-menopausal female or a female with an estrogen deficiency condition are about 50 pg/ml in late follicular phase after administering the female a regimen of the present invention.

In some embodiments, the free estradiol levels of a peri-menopausal female or a female with an estrogen deficiency condition are about 40 pg/ml to about 450 pg/ml in luteal phase after administering the female a regimen of the present invention. In other embodiments, the free estradiol levels of a peri-menopausal female or a female with an estrogen deficiency condition are about 50 pg/ml to about 400 pg/ml, about 50 pg/ml to about 300 pg/ml, about 50 pg/ml to about 250 pg/ml, about 50 pg/ml to about 200 pg/ml, about 50 pg/ml to about 150 pg/ml, about 50 pg/ml to about 130 pg/ml, about 50 pg/ml to about 110 pg/ml, about 50 pg/ml to about 90 pg/ml or about 50 pg/ml to about 70 pg/ml in luteal phase after administering the female a regimen of the present invention. In some embodiments, the free estradiol levels of a peri-menopausal female or a female with an estrogen deficiency condition are about 50 pg/ml in luteal phase after administering the female a regimen of the present invention.

The present invention is also directed to a method of treating amenorrhea in a female in need thereof by administering to the female a regimen of the present invention, disclosed herein.

The terms “secondary amenorrhea” and “amenorrhea” as used herein refer to the absence of menstrual periods in a female after menarche (i.e., in a female whose periods were regularly established before menstruation fails to occur).

Amenorrhea affects 2% to 5% of all women of childbearing age in the United States. Female athletes, especially young women, may be more likely to have amenorrhea. While exercise or physical activity itself does not cause amenorrhea, it is more likely to occur in women who exercise very intensely or who increase the intensity of exercise rapidly. Women who engage in sports associated with lower body weight, such as ballet dancing or gymnastics, are more likely to develop amenorrhea than women in other sports.

The present invention is also directed to a method of treating amenorrhea and stimulating estrogen production in a female in need thereof by administering to the female a regimen of the present invention, disclosed herein.

The present invention is also directed to a method treating anovulation in a female in need thereof by administering to the female a regimen of the present invention, disclosed herein.

The term “anovulation” as used herein refers to the absence of ovulation. Ovulation as used herein refers to the formation of ova or eggs in the ovary, and the discharge of the same. Causes of anovulation that are anticipated by the invention are, but are not limited to, excessive exercise, excessive weight loss, stress, drugs, estrogen and progesterone imbalances, a malfunctioning corpulsteum, congenital adrenal hyperplasia, premature ovarian failure, and hyperprolactinemia.

The present invention is also directed to a method of treating or preventing a disease, disorder, or a symptom associated with deficient endogenous levels of estrogen in a female in need thereof by administering to the female a regimen of the present invention, disclosed herein.

Low levels of estrogen, irrespective of the cause, lead to an overall decreased quality of life for women. Symptoms, diseases and disorders range from merely being inconvenient to life threatening. The present invention is also directed to treating all physiological and psychological signs of estrogen deficiency.

The present invention is also directed to treating transient symptoms of estrogen deficiency, such as vasomotor signs and psychological symptoms. Vasomotor signs comprise but are not limited to hot flushes, sweating attacks such as night sweats, and palpitations. Psychological symptoms of estrogen deficiency comprise, but are not limited to, insomnia and other sleep disorders, poor memory, loss of confidence (or self-esteem), mood changes, anxiety, loss of libido, difficulties in concentration, difficulty in making decisions, diminished energy and drive, irritability, and crying spells.

The treatment of the aforementioned symptoms can be associated with the peri-menopausal phase of a woman's life or after, sometimes long after menopause. It is anticipated that the invention is applicable to these and other transient symptoms during the peri-menopausal phase. Moreover, the aforementioned symptoms can be alleviated if the cause of the estrogen deficiency is hypogonadism or primary ovarian failure.

The invention can be used for the treatment of permanent effects of estrogen deficiency. Permanent effects comprise physical changes such as urogenital atrophy, atrophy of the breasts, changes in hair distribution, thickness of hair, changes in skin condition and osteoporosis.

Urogenital atrophy, conditions associated with it such as vaginal dryness, increase in vaginal pH and subsequent changes in flora, or events which lead to such atrophy, such as decreases in vascularity, fragmentation of elastic fibres, fusion of collagen fibres, or decreases in cell volume are symptoms thought to be particularly relevant to the present invention. Furthermore, the invention is thought to be relevant to other urogenital changes associated estrogen deficiency such as decreases in the length and/or diameter of the vagina, decreases mucus production, changes in cell population, decreases in glycogen production, decreases in growth of lactobacilli or increases in growth of streptococci, staphylococci, or coliform bacilli. Other associated changes that are thought to be preventable, by the invention are those that may render the vagina susceptible to injury or infection, such as exudative discharges, vaginitis, and dyspareunia. Furthermore, infections of the urinary tract and incontinence are other common symptoms associated with lowered estrogen levels.

The present invention is also directed to a method of increasing fertility in a female in need thereof by administering to he female a regimen of the present invention, disclosed herein. The female can be, but is not limited to, a female of child bearing age or a peri-menopausal female.

It has been observed clinically that women who are taking oral contraceptives for anovulation often conceive when pills are missed, or shortly after discontinuing oral contraceptive treatment, most likely due to a “rebound effect” occurring in the ovary at least for a short period of time. Suppression of ovarian activity using oral contraceptive pills for 2-6 months may result in decreases in early follicular ovarian androgen production and LH and estradiol levels. Increased androgen levels have been shown to have adverse effects on folliculogenesis. These endocrine changes in the early follicular phase may be responsible for improved ovarian response to clomiphene or other treatments for anovulatory infertility. See Brannigan, E. F., and Estes, M. A., Am. J. Obstet. Gynecol. 188: 1424-1430 (2003).

### Dosages and Formulations

In the regimens of the present invention, the daily dosage of the estrogen that is administered with the progestin is equivalent to about 10 μg to about 50 μg of ethinyl estradiol. In some aspects of the invention, the daily dosage of estrogen is equivalent to about 10 μg to about 25 μg of ethinyl estradiol. In other aspects of the invention, the daily dosage of estrogen is equivalent to about 25 μg to about 40 μg of ethinyl estradiol. In yet other aspects of the invention, the daily dosage of estrogen is equivalent to about 10 μg to about 30 μg of ethinyl estradiol. In yet other aspects of the invention, the daily dosage of estrogen is equivalent to about 15 μg to about 30 μg of ethinyl estradiol.

In some aspects of the invention, the daily dosage of estrogen that is administered with the progestin in the regimens of the present invention is equivalent to about 10 μg, about 15 μg, about 20 μg, about 25 μg, about 30 μg, about 35 μg, about 40 μg, about 45 μg, or about 50 μg of ethinyl estradiol.

The daily dosage of the progestin administered in regimens of the present invention is equivalent to about 0.05 mg to about 0.5 mg of levonorgestrel. In some aspects of the invention, the daily dosage of progestin is equivalent to about 0.05 mg to about 0.25 mg of levonorgestrel. In other aspects of the invention, the daily dosage of progestin is equivalent to about 0.05 mg to about 0.20 mg of levonorgestrel.

In some aspects, the daily dosage of the progestin administered in regimens of the present invention is equivalent to about 0.05 mg, about 0.10 mg, about 0.15 mg, about 0.20 mg, or about 0.25 mg of levonorgestrel.

The daily dosage of estrogen administered during the unopposed estrogen interval in the bridged regimen is equivalent to about 5 μg to about 50 μg of ethinyl estradiol. In some embodiments of the invention, the daily dosage amount of estrogen is equivalent to about 5 μg to about 30 μg of ethinyl estradiol, or is equivalent to about 5 μg to about 25 μg of ethinyl estradiol. In other embodiments, the daily dose of estrogen is equivalent to about 10 pg to about 25 μg of ethinyl estradiol, or is equivalent to about 10 μg to about 20 μg of ethinyl estradiol. In yet other embodiments of the invention, the daily dose of estrogen is equivalent to about 5 μg to about 15 pg of ethinyl estradiol.

In some aspects of the invention, the daily dosage of estrogen administered during the unopposed estrogen interval in the bridged regimen is equivalent to about 5 μg, about 10 μg, about 15 μg, about 20 μg, about 25 μg, or about 30 μg of ethinyl estradiol.

In some aspects of the present invention, the estrogen and progestin of the regimens of the present invention can be ethinyl estradiol and levonorgestrel, respectively, although other suitable estrogens and progestins can be employed. The weight ratio of estrogen and progestin can be about 1:0.2 to about 1:300. In some aspects of the invention, the weight ratio of estrogen and progestin is about 1:1 to about 1:50. In other aspects of the invention, the weight ratio of estrogen and progestin is about 1:1 to about 1:10. For example, the daily amount of ethinyl estradiol is about 10 μg to about 30 μg and the daily amount of levonorgestrel is about 0.05 mg to about 0.2 mg.

As used herein, the term “about” refers to plus or minus 10% of the indicated number. For example, “about 90%” refers to 81% to 99%.

The values given above are for ethinyl estradiol and levonorgestrel, and if a different estrogen or progestin is employed, an adjustment in the amount based on the relative potency or activity can be made. Correlations in potency among the various estrogens and among the various progestins are known See, for example, EP 0 253 607, which is hereby incorporated in its entirety by reference. For example, in a contraceptive regimen, 30 μg of ethinyl estradiol is roughly equivalent to about 60 μg of mestranol or about 2,000 μg of 17β-estradiol. Similarly, 0.050 mg of levonorgestrel is roughly equivalent to about 0.175 mg of norethindrone acetate, about 0.050 mg of desogestrel, about 0.050 mg 3-ketodesogestrel, about 0.035 mg of gestodene, or about 0.100 mg of norgestrel. It should be understood that when norgestrel is used in place of levonorgestrel, its concentration is twice that of levonorgestrel. Norgestrel (dl-norgestrel) is a racemic mixture of optically active isomers, while levonorgestrel is one of the optically active isomers present in norgestrel.

Equivalent concentrations of estrogens and of progestins can be determined using either in vitro or in vivo assay methods. See, for example, Kuhl, H., Drugs 51(2):188-215 (1996); Philibert, D., et al., Gynecol. Endocrinol. 13:316-326 (1999); and Lundeen, S., et al., J. Steroid Biochem. Molec. Biol. 78:137-143 (2001), in which the relative potencies of various progestins are compared using both in vitro and in vivo test assays. See also, for example, Dickey, R. P., “Contraceptive Therapy,” OBG Management Supplement (October 2000), pp. 2-6. Each of these documents is hereby incorporated by reference in its entirety.

For example, various combinations of progestin and estrogen that have been used in oral contraceptives are shown in Table 1.

TABLE 1
Combinations of Progestin and Estrogen

| | | Norethindrone | | | EE | |
|---|---|---|---|---|---|---|
| | Dose | Equivalent* | | Dose | Equivalent* | P/E |
| Progestin | (mg) | Dose (mg) | Estrogen | (mg) | Dose (mg) | Ratio |
| Norethynodrel | 9.85 | 9.85 | Mestranol | 0.150 | 0.105 | 93.810 |
| | 5.00 | 5.00 | | 0.075 | 0.053 | 95.238 |
| | 2.50 | 2.50 | | 0.036 | 0.025 | 99.206 |
| | 2.50 | 2.50 | | 0.100 | 0.070 | 35.714 |
| Norethindrone | 10.00 | 10.00 | Mestranol | 0.060 | 0.042 | 238.095 |
| | 2.00 | 2.00 | | 0.100 | 0.070 | 28.571 |
| | 1.00 | 1.00 | | 0.050 | 0.035 | 28.571 |
| | 1.00 | 1.00 | | 0.080 | 0.056 | 17.857 |
| Norethindrone | 1.00 | 1.00 | Ethinyl | 0.050 | 0.050 | 20.000 |
| acetate | 1.00 | 1.00 | Estradiol | 0.035 | 0.035 | 28.571 |
| | 0.50 | 0.50 | (EE) | 0.035 | 0.035 | 14.286 |
| | 0.40 | 0.40 | | 0.035 | 0.035 | 11.429 |
| Norethindrone | 2.50 | 2.50 | EE | 0.050 | 0.050 | 50.000 |
| acetate | 1.00 | 1.00 | | 0.050 | 0.050 | 20.000 |
| | 0.60 | 0.60 | | 0.030 | 0.030 | 20.000 |
| | 1.50 | 1.50 | | 0.030 | 0.030 | 50.000 |
| | 1.00 | 1.00 | | 0.020 | 0.020 | 50.000 |
| Ethynodiol diacetate | 1.00 | 1.00 | Mestranol | 0.100 | 0.100 | 14.286 |
| Ethynodiol diacetate | 1.00 | 1.00 | EE | 0.050 | 0.050 | 20.000 |
| | 1.00 | 1.00 | | 0.035 | 0.035 | 28.571 |
| dl-Norgestrel | 0.50 | 0.75 | EE | 0.050 | 0.050 | 10.000 |
| | 0.30 | 0.45 | | 0.030 | 0.030 | 10.000 |
| Levonorgestrel | 0.10 | 0.35 | EE | 0.020 | 0.020 | 5.000 |
| | 0.15 | 0.52 | | 0.030 | 0.030 | 5.000 |

*Equivalencies: 0.050 mg Mestranol = approximately 0.035 mg Ethinyl estradiol (EE); and 0.10 mg dl-Norgestrel = approximately 0.15 mg Norethindrone

Each block in Table 1 describes a specific combination of progestin and estrogen, e.g., norethynodrel and mestranol, and within each block older combinations are listed first, with successively newer combinations following.

Suitable progestins for use in the present invention include, but are not limited to, natural and synthetic compounds having progestational activity, such as, for example, progesterone, chlormadinone acetate, norethindrone, cyproterone acetate, norethindrone acetate, desogestrel, levonorgestrel, drospirenone, trimegestone, norgestrel, norgestimate, norelgestromin, etonogestrel, gestodene, and other natural and/or synthetic gestagens. Prodrugs of suitable progestins can also be used in a regimen of the present invention.

The expression “prodrug” denotes a derivative of a known direct acting drug, which derivative has enhanced delivery characteristics and therapeutic value as compared to the drug and is transformed into the active drug by an enzymatic or chemical process. Ethynodiol diacetate, which is converted in vivo to norethindrone, is an example of a progestin prodrug that can be used in the present invention. Additional examples of progestin prodrugs include, but are not limited to, norgestimate (which is converted in vivo to 17-deacetyl norgestimate, also known as norelgestromin), desogestrel (which is converted in vivo to 3-keto desogestrel, also known as etonogestrel), and norethindrone acetate (which is converted in vivo to norethindrone).

Suitable estrogens in the present invention include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); mestranol; agonistic anti-estrogens; and selective estrogen receptor modulators. Prodrugs of suitable estrogens can also be used in a regimen of the present invention. Examples of estrogen prodrugs that can be used in the present invention include, but are not limited to, estradiol acetate (which is converted in vivo to 17β-estradiol) and mestranol (which is converted in vivo to ethinyl estradiol).

The estrogen and progestin are administered in the conventional manner by any route where they are active. For example, administration can be by, but is not limited to, oral, parenteral, subcutaneous, intravenous, intramuscular, intraperitoneal, transdermal, buccal, or ocular routes, or intravaginally, by inhalation, by depot injections, or by hormone implants. Thus, modes of administration for the estrogen and progestin (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams.

Pharmaceutical formulations containing the estrogen and/or progestin and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder comprising an effective amount of the estrogen and/or progestin as taught in this invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance. For example, “Modern Pharmaceutics,” Banker & Rhodes, Marcel Dekker, Inc. (1979); and “Goodman & Gilman's The Pharmaceutical Basis of Therapeutics,” MacMillan Publishing Co., New York, 6th ed. (1980) can be consulted.

Most estrogens and progestins are orally active and this route of administration can be used in the invention. Accordingly, administration forms can include, but are not limited to, tablets, dragees, capsules and pills, which contain the estrogen and the progestin and one or more suitable pharmaceutically acceptable carriers.

For oral administration, the estrogen and/or progestin can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the estrogen and/or progestin in admixture with filler such as, e.g., lactose, binders such as, e.g., starches, and/or lubricants such as, e.g., talc or magnesium stearate and, optionally, stabilizers. In soft capsules, the estrogen and/or progestin can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the estrogen and/or progestin compositions can take the form of, e.g., tablets or lozenges formulated in a conventional manner.

For administration by inhalation, the estrogen and/or progestin are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The estrogen and/or progestin can be formulated for parenteral administration by injection, e.g., by bolus injection or continuous infusion. The estrogen and/or progestin can be administered by continuous infusion subcutaneously over a period of about 15 minutes to about 24 hours. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

The estrogen and/or progestin can also be formulated in rectal compositions such as suppositories or retention enemas, e.g., containing conventional suppository bases such as cocoa butter or other glycerides.

In addition to the formulations described previously, the estrogen and/or progestin can also be formulated as a depot preparation. Such long acting formulations can be administered by implantation (for example subcutaneously or intramuscularly) or by intramuscular injection. Depot injections can be administered for about 1 to about 6 months or longer intervals. Thus, for example, the estrogen and/or progestin can be formulated with suitable polymeric or hydrophobic materials (for example as an emulsion in an acceptable oil) or ion exchange resins, or as sparingly soluble derivatives, for example, as a sparingly soluble salt.

For transdermal administration, the estrogen and progestin can be applied by any transdermal, therapeutic system that is consequently supplied to the organism, such as, for example, as a transdermal patch, transdermal cream or plaster. For example, the estrogen and/or progestin can be formulated as a transdermal patch. The preparation and use of transdermal patches are well known to those of skill in the art and are available in different designs, including matrix-type or reservoir-type designs. In addition to the estrogen and/or progestin, transdermal patches can contain additional components such as penetration-enhancing agents and/or additional excipients that are conventionally employed, e.g., carriers, gelling agents, suspending agents, dispersing agents, preservatives, stabilizers, wetting agents, emulsifying agents, and the like.

For vaginal administration, the estrogen and/or progestin can be formulated as vaginal gels, creams, tampons, suppositories, vaginal rings, intrauterine devices and the like. The preparation of each of these formulations is well known to those of skill in the art.

The estrogen and progestin can also be administered according to the regimens of the present invention in combination with other pharmaceutically active agents or compounds, including, for example, glucocorticoids such as vitamin D or vitamin D analogues; and/or minerals, e.g., calcium. For example, the estrogen and/or progestin can be administered with vitamin D and/or calcium as a method of maintaining or preventing loss of bone density. The form of vitamin D and of calcium used in the present invention would be well known to those of skill in the art, as would the amount. For example, calcium can be administered in the form of calcium carbonate, at a daily dosage level of about 500 mg.

Examples of other pharmaceutically active agents that can be administered with estrogen and progestin according to the regimens of the present invention include, but are not limited to, one or more of the B complex vitamins, such as vitamin B3 (niacin (i.e., nicotinic acid and/or nicotinamide)), vitamin B6 and/or vitamin B12; iron (e.g., ferrous iron, such as, e.g., ferrous sulfate, ferrous fumarate, ferrous gluconate, or an iron glycine amino acid chelate); bisphosphonates (e.g., alendronate); teriparatide (e.g., FORTEO™); and SERMs (selective estrogen receptor modulators, e.g., raloxifene).

The estrogen and/or progestin can also be administered with an antidepressant, such as, for example, a selective serotonin reuptake inhibitor (SSRI), a tricyclic antidepressant or anxiolytic, or any antidepressant known to one of skill in the art. Suitable antidepressants include, but are not limited to, alprazolam (XANAX®, Pharmacia and Upjohn, a division of Pfizer Inc., New York, N.Y.), clomipramine (ANAFRANIL®, Mallinckrodt Inc., St. Louis, Mo.), fluoxetine (PROZAC®, Eli Lilly and Co., Indianapolis, Ind.), paroxetine (PAXIL®, GlaxoSmithKline Corporation, Research Triangle Park, N.C.), sertraline (ZOLOFT®, Pfizer Inc., New York, N.Y.), nefazodone, and venlafaxine (EFFEXOR®, Wyeth Pharmaceuticals Inc., Philadelphia, Pa.).

The additional pharmaceutically active agents can be administered using any suitable modes of administration, including, but not limited to, parenteral, oral, buccal, rectal, subcutaneous, intravenous, intramuscular, intranasal, transdermal modes of administration, and by inhalation. In some aspects of the invention, the additional active agent is administered using the same mode of administration as the estrogen and/or progestin. For example, the additional active agent and the estrogen and/or progestin are administered together using the same mode of administration, either in the same dosage form (e.g., transdermally, using the same vaginal ring) or, alternatively, in two different dosage forms (e.g., as two separate vaginal creams). In other aspects, the additional active agent is administered using a different mode of administration, e.g., the estrogen and/or progestin are administered transdermally, using a transdermal delivery device such as a vaginal ring, and the additional active agent, e.g., an antidepressant, is administered orally, in the form of a pill or tablet.

The dosage of the additional active agent can be determined readily by one of skill in the medical arts and will depend upon the condition or disorder to be treated, the physiological effect desired, and the mode of administration. For example, the amount of antidepressant administered with the estrogen, or with the estrogen and progestin, depending on the antidepressant used, is about 0.75 to about 2 mg/24 hours, about 10 to about 20 mg/24 hours, or about 50 to about 100 mg/24 hours. Thus, in some aspects of the invention, the estrogen and progestin are administered with about 5 mg to about 120 mg/24 hours of fluoxetine hydrochloride. As another example, calcium administered with the estrogen and progestin can be in the form of calcium carbonate, at a daily dosage level of about 500 mg.

The regimens of the present invention can be produced in the form of a kit or package containing the dosage units to be administered according to a regimen of the present invention, the multiple dosage units can be optionally arranged for sequential administration.

For example, in some embodiments of the present invention, the kit contains 21 or more tablets for oral administration, each tablet containing a combination of estrogen and progestin and intended for ingestion on successive days, and 11 or more placebo tablets (hormone free), each tablet containing neither estrogen nor progestin and intended for ingestion on successive days. In each tablet that contains the combination of estrogen and progestin, estrogen can be present in an amount equivalent to about 10 μg to about 50 μg of ethinyl estradiol, and progestin can be present in an amount equivalent to about 50 μg to about 0.5 mg levonorgestrel. Administration is daily for at least 21 consecutive days using tablets containing the both the estrogen and the progestin, immediately followed by administration that is daily for at least 11 consecutive days using the hormone-free placebo tablets.

For example, administration can be for 21 to 119 consecutive days, using tablets containing both estrogen and progestin, immediately followed by administration for at least 11 consecutive days, using hormone-free placebo tablets. In yet another example, administration can be for 21 to 91 consecutive days, using tablets containing both estrogen and progestin, immediately followed by administration for at least 11 consecutive days, using hormone-free placebo tablets.

In another example, regimens of the present invention can be provided in kit form containing, e.g., for a 98-day regimen, 84 tablets, each tablet containing estrogen and progestin, intended for ingestion on successive days, immediately followed by 14 hormone-free placebo tablets, intended for ingestion on successive days.

In alternative embodiments of the present invention, the bridged regimens can be provided in kit form containing at least 21 tablets containing estrogen and progestin, intended for ingestion on successive days, immediately followed by 1 to 14 tablets containing estrogen without progestin, and then immediately followed by 11 to 91 placebo tablets containing neither estrogen nor progestin, intended for ingestion on successive days. In each tablet that contains the combination of estrogen and progestin, estrogen can be present in an amount equivalent to about 10 μg to about 50 μg of ethinyl estradiol, and progestin can be present in an amount equivalent to about 50 μg to about 0.5 mg levonorgestrel. In each tablet that contains estrogen without progestin, estrogen is present in an amount equivalent to about 5 μg to about 50 μg of ethinyl estradiol. Administration is daily for at least 21 consecutive days using the tablets containing the both the estrogen and the progestin, immediately followed by administration that is daily for 1 to 14 consecutive days using the tablets containing estrogen without progestin, and then immediately followed by administration that is daily for, e.g., 11 to 91 consecutive days, using placebo tablets containing neither estrogen or progestin. For example, administration can be for 21-119 days, using tablets containing both estrogen and progestin, immediately followed by administration for 1-14 consecutive days of estrogen without progestin, using tablets containing estrogen without progestin, and then immediately followed by administration for 11-91 consecutive days of neither estrogen nor progestin, using placebo tablets containing neither estrogen nor progestin. As yet another example, administration can be for 21-91 consecutive days, using tablets containing both estrogen and progestin, immediately followed by administration for 1-14 consecutive days of estrogen without progestin, using tablets containing estrogen without progestin, and then immediately followed by administration for 11-91 consecutive days of neither estrogen nor progestin, using placebo tablets containing neither estrogen nor progestin.

In another example, the bridged regimen can be provided in kit form containing, for a 39-day regimen, 25 tablets, each tablet containing estrogen and progestin, intended for ingestion on successive days, immediately followed by 3 tablets, each tablet containing estrogen without progestin, and then immediately followed by 11 placebo tablets, each tablet containing neither estrogen or progestin, intended for ingestion on successive days. In other aspects of the invention, the bridged regimen can be provided in kit form containing 25 tablets, each tablet containing both the estrogen and the progestin, intended for ingestion on successive days, and 3 tablets, each tablet containing both estrogen and an antidepressant, e.g., fluoxetine hydrochloride, immediately followed by 11 placebo tablets, each tablet containing a placebo with neither estrogen or progestin, intended for ingestion on successive days.

The kits of the present invention can optionally contain instructions associated with the dosage units of the kits. Such instructions can be in a form prescribed by a governmental agency regulating the manufacture, use or sale of pharmaceuticals or biological products, which notice reflects approval by the agency of the manufacture, use or sale for human administration to treat a condition or disorder. The instructions can be in any form which conveys information on the use of the dosage units in the kit according to the methods of the invention. For example, the instructions can be in the form of printed matter, or in the form of a pre-recorded media device.

“Printed matter” can be, for example, one of a book, booklet, brochure or leaflet. The printed matter can describe the use of the dosage units of the kit according to the regimens of the present invention. Possible formats include, but are not limited to, a bullet point list, a list of frequently asked questions (FAQ) or a chart. Additionally, the information to be imparted can be illustrated in non-textual terms using pictures, graphics or other symbols.

“Pre-recorded media device” can be, for example, a visual media device, such as a videotape cassette, a DVD (digital video disk), filmstrip, 35 mm movie or any other visual media device. Alternately, pre-recorded media device can be an interactive software application, such as a CD-ROM (compact disk-read only memory) or floppy disk. Alternately, pre-recorded media device can be, for example, an audio media device, such as a record, audiocassette or audio compact disk. The information contained on the pre-recorded media device can describe the proper use of the dosage units in the kit for the treatment of one or more of the conditions or disorders as described herein.

In addition to instructions, the kit can optionally contain a planner. A “planner” can be, for example, a weekly, a monthly, a multi-monthly, a yearly, or a multi-yearly planner. The planner can be used as a diary to monitor dosage amounts, to keep track of dosages administered, or to prepare for future events wherein taking the dosages of the kit can be difficult. Alternately, the planner can be a calendar which will provide a means to monitor when a dosage has been taken and when it has not been taken. This type of planner will be particularly useful for patients having unusual schedules for administering medication to themselves. One skilled in the art will appreciate the variety of planning tools that would be appropriate for use with the present invention.

The kit can also include a container for storing the other components of the kit. The container can be, for example, a bag, box, envelope or any other container that would be suitable for use in the present invention. The container can be large enough to accommodate each component and/or any administrative devices that can be necessary for use of the dosage units of the kit according to the methods of the present invention. However, in some cases, it can be desirable to have a smaller container which can be hidden in a patient's pocketbook, briefcase or pocket.

The present invention is also directed to a method of delivery of a regimen disclosed herein according to the methods of the present invention (e.g., a method of stimulating estrogen production) to a patient in need thereof, the method comprising (a) registering in a computer readable medium the identity of a physician permitted to prescribe the regimen; (b) providing the patient with counseling information concerning the risks attendant to the regimen; (c) obtaining informed consent from the patient to receive the regimen despite the attendant risks; (d) registering the patient in a computer readable medium after obtaining their informed consent; and (e) permitting the patient access to the regimen.

The drug delivery methods of the present invention involve, inter alia, registering in a computer readable storage medium physicians who are qualified to prescribe the regimen to be used according to the methods of the present invention. Once registered in the computer readable storage medium, the physician can be eligible to prescribe a regimen according to the methods of the invention to a patient in need thereof. Generally speaking, in order to become registered in the computer readable storage medium, the physician can be required to comply with various aspects of, for example, providing patient education and counseling. Registration of the physician in the computer readable storage medium can be achieved by providing the physician, for example, by mail, facsimile transmission, or on-line transmission, with a registration card or form, preferably together with educational materials concerning the regimen. The physician can complete the registration card or form by providing information requested therein, and the registration card or form can be returned to the manufacturer or distributor of the regimen, or other authorized recipient of the registration materials, for example, by mail, facsimile transmission or on-line transmission. The physician's information in the registration card or form is then entered into the computer readable storage medium. Suitable computer readable storage media which can be employed for registration of the physicians (as well as patients, as discussed below) will be apparent to one of ordinary skill in the art, once in possession of the teaching of the present application.

In the course of examination of a patient, the physician may determine that administration of one of the regimens of the present invention is appropriate for the patient, or the physician may determine that the patient's condition (e.g., the patient may be suffering from amenorrhea) can be improved by the administration of one of the regimens of the present invention according to the methods of the present invention. Prior to prescribing the regimen, the physician can counsel the patient, for example, on the various risks and benefits associated with the regimen. The patient can be provided full disclosure of all the known and suspected risks associated with the regimen. Such counseling can be provided verbally, as well as in written form. In some embodiments, the physician can provide the patient with literature materials on the regimen, such as product information, educational materials, and the like.

In addition to receiving counseling on the risks attendant to administration of the regimens disclosed herein, the methods of the present invention further require the patient to fill out an informed consent form which is signed by the patient. Upon the completion of the informed consent form, the patient can be registered in a computer readable storage medium. The computer readable storage medium in which the patient is registered can be the same as, or different from, the computer readable storage medium in which the physician is registered.

The registration into one or more computer readable storage media of the physician and patient, according to the methods describe herein, provides a means to monitor and authorize access to the regimen administered according to the methods of the present invention. Thus, the computer readable storage medium can serve to deny access to patients who fail to abide by the methods of the present invention. In some embodiments, access to the regimen is in the form of a prescription, wherein the prescribing physician is registered in a computer readable storage medium, has provided counseling to the patient concerning the attendant risks of the regimen, and has obtained informed consent from the patient, prior to prescribing the regimen to the patient in need thereof according to the methods of the present invention.

The present invention is also directed to methods of educating consumers about the use of the regimens of the present invention, the method comprising distributing the regimen with consumer information at a point of sale. In some embodiments, the distribution will occur at a point of sale having a pharmacist or healthcare provider.

As used herein, the term “consumer information” can include, but is not limited to, an English language text, non-English language text, visual image, chart, telephone recording, website, and access to a live costumer service representative. In some embodiments, consumer information will provide directions for use of the regimens according to the methods of the present invention, appropriate age use, indication, contraindications, appropriate dosing, warnings, telephone number of website address. In some embodiments, the method further comprises providing professional information to relevant persons in a position to answer consumer questions regarding use of the disclosed regimens according to the methods of the present invention.

As used herein, the term “professional information” includes, but is not limited to, information concerning the regimen when administered according to the methods of the present invention that is designed to enable a healthcare professional to answer costumer questions.

A “relevant person,” as used herein, includes, for example, a physician, physician assistant, nurse practitioner, pharmacist and customer service representative.

All of the various aspects, embodiments and options described herein can be combined in any and all variations. The following examples are illustrative, but not limiting, of the method and compositions of the present invention. Other suitable modifications and adaptations of the variety of conditions and parameters normally encountered and obvious to those skilled in the art are within the spirit and scope of the invention. Thus, the breadth and scope of the present invention should not be limited by any of the below-described exemplary embodiments, but should be defined only in accordance with the following claims and their equivalents.

### Example 1

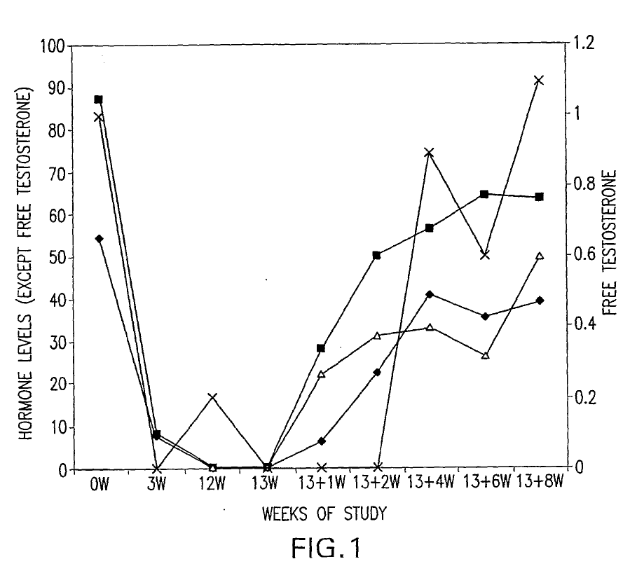

In this study, about 30 healthy, premenopausal women were enrolled and treated with 84 consecutive daily doses of levonorgestrel/ethinyl estradiol tablet (150 μg/30 μg) followed by 7 consecutive daily doses of a 30 μg ethinyl estradiol tablet. Blood concentrations of endogenous hormones (follicle stimulating hormone (FSH), luteinizing hormone (LH), estradiol, total testosterone, and free testosterone) were measured at various times during treatment and after completion of treatment. Although the women in this study were supposed to be premenopausal and not amenorrheic, the endogenous hormone levels for one volunteer were found to be well within the postmenopausal ranges prior to receiving any study medication. Menopause is characterized by very low estradiol levels (<20 pg/ml) and very high FSH levels (>40 mIU/ml). This subject's estradiol levels remained in the postmenopausal range throughout the 13 week treatment phase of the study. One week after discontinuing the medication, her estradiol levels increased above the menopausal range, and continued to increase to about 50 pg/ml at 8 weeks following discontinuing the study medication (FIG. 1).

Having now fully described this invention, it will be understood to those of ordinary skill in the art that the same can be performed within a wide and equivalent range of conditions, formulations, and other parameters without affecting the scope of the invention or any embodiment thereof.

All documents, e.g., scientific publications, patents, patent applications and patent publications recited herein are hereby incorporated by reference in their entirety to the same extent as if each individual document was specifically and individually indicated to be incorporated by reference in its entirety. Where the document cited only provides the first page of the document, the entire document is intended, including the remaining pages of the document.

What is claimed is:

1. A method of stimulating estrogen production in a pre-menopausal or peri-menopausal female in need thereof, the method comprising administering to the female a combination of estrogen and progestin for a period of 21 or more consecutive days, immediately followed by a hormone-free period of 11 or more consecutive days.

2. The method of claim 1, wherein the combination of estrogen and progestin is administered for a period of 21 to 119 consecutive days.

3. The method of claim 1, wherein the combination of estrogen and progestin is administered for a period of 21 to 91 consecutive days.

4. The method of claim 1, wherein the hormone-free period is 11 days to 91 days.

5. The method of claim 1, wherein the estrogen that is administered in combination with the progestin for a period of 21 or more consecutive days is administered in a daily amount equivalent to about 10 μg to about 50 μg of ethinyl estradiol, and the progestin that is administered in combination with estrogen for a period of 21 or more consecutive days is administered in a daily amount equivalent to about 0.05 mg to about 0.5 mg of levonorgestrel.

6. The method of claim 1, wherein the estrogen that is administered in combination with the progestin for a period of 21 or more consecutive days is administered by an oral tablet.

7. The method of claim 1, wherein the progestin that is administered in combination with the estrogen for a period of 21 or more consecutive days is administered by an oral tablet.

8. The method of claim 1, wherein the combination of progestin and estrogen that is administered for a period of 21 or more consecutive days is administered by an oral tablet.

9. The method of claim 1, wherein the hormone-free period is immediately followed by a combination of estrogen and progestin for a period of 21 or more consecutive days, immediately followed by a hormone-free period of 11 or more consecutive days.

10. The method of claim 1, further comprising the female having free estradiol levels of about 40 to about 150 pg/ml during early follicular phase of a female's menstrual cycle following the administration of estrogen and progestin and during the hormone-free period.

11. The method of claim 1, further comprising the female having free estradiol levels of about 40 to about 350 pg/ml during late follicular phase of a female's menstrual cycle following the administration of estrogen and progestin and during the hormone-free period.

12. The method of claim 1, further comprising the female having free estradiol levels of about 150 to about 750 pg/ml during luteal phase of a female's menstrual cycle following the administration of estrogen and progestin and during the hormone-free period.

13. A method of stimulating estrogen production in a pre-menopausal or peri-menopausal female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

14. The method of claim 13, wherein the combination of estrogen and progestin is administered for a period of 21 to 119 consecutive days.

15. The method of claim 13, wherein the combination of estrogen and progestin is administered for a period of 21 to 91 consecutive days.

16. The method of claim 13, wherein the hormone-free period is 14 days to 91 days.

17. The method of claim 13, wherein the estrogen that is administered in combination with the progestin for a period of 21 or more consecutive days is administered in a daily amount equivalent to about 10 μg to about 50 μg of ethinyl estradiol, and the progestin that is administered in combination with estrogen for a period of 21 or more consecutive days is administered in a daily amount equivalent to about 0.05 mg to about 0.5 mg of levonorgestrel.

18. The method of claim 13, wherein the estrogen without progestin that is administered for a period of 1 to 14 consecutive days is administered in a daily amount equivalent to about 5 μg to about 50 μg of ethinyl estradiol.

19. The method of claim 13, wherein the estrogen that is administered in combination with the progestin for a period of 21 or more consecutive days is administered by an oral tablet.

20. The method of claim 13, wherein the progestin that is administered in combination with the estrogen for a period of 21 or more consecutive days is administered by an oral tablet.

21. The method of claim 13, wherein the combination of progestin and estrogen that is administered for a period of 21 or more consecutive days is administered by an oral tablet.

22. The method of claim 13, wherein the hormone-free period is immediately followed by a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

23. A method of treating estrogen deficiency in a female in need thereof, the method comprising administering to the female a combination of estrogen and progestin for a period of 21 or more consecutive days, immediately followed by a hormone-free period of 11 or more consecutive days.

24. A method of treating estrogen deficiency in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

25. A method of restoring estrogen to a pre-menopausal level in a female in need thereof, the method comprising administering to the female a combination of estrogen and progestin for a period of 21 or more consecutive days, immediately followed by a hormone-free period of 11 or more consecutive days.

26. A method of restoring estrogen to a pre-menopausal level in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of more 11 to 91 consecutive days.

27. A method of treating amenorrhea in a female in need thereof, the method comprising administering to the female a combination of estrogen and progestin for a period of 21 or more consecutive days, immediately followed by a hormone-free period of 11 or more consecutive days.

28. A method of treating amenorrhea in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

29. A method of treating anovulation in a female in need thereof, the method comprising administering to the female a combination of estrogen and progestin for a period of 21 or more consecutive days, immediately followed by a hormone-free period of 11 or more consecutive days.

30. A method of treating anovulation in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

31. A method of treating or preventing a disease, a disorder, or a symptom associated with deficient endogenous levels of estrogen in a female in need thereof, the method comprising administering to the female a combination of estrogen and progestin for a period of 21 or more consecutive days, immediately followed by a hormone-free period of 11 or more consecutive days.

32. A method of treating or preventing a disease, a disorder, or a symptom associated with deficient endogenous levels of estrogen in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

33. A method of increasing fertility in a female in need thereof, the method comprising administering to the female a combination of estrogen and progestin for a period of 21 or more consecutive days, immediately followed by a hormone-free period of 11 or more consecutive days.

34. A method of increasing fertility in a female in need thereof, the method comprising administering to the female a combination of estrogen and progestin for a period of 21 or more consecutive days, immediately followed by administration of estrogen without progestin for a period of 1 to 14 consecutive days, and then immediately followed by a hormone-free period of 11 to 91 consecutive days.

35. A pharmaceutical kit comprising:
a) 21 or more daily doses of estrogen;
b) 21 or more daily doses of progestin;
c) 11 or more daily doses of a placebo pill; and
d) a label comprising a direction for administering the placebo pill after the estrogen and progestin.

36. The pharmaceutical kit of claim 35, wherein the daily doses of estrogen are in combination with the daily doses of progestin.

37. The pharmaceutical kit of claim 36, further comprising 1 to 14 daily doses of estrogen without progestin.

38. The pharmaceutical kit of claim 35, comprising 11 to 91 daily doses of a placebo pill.

39. The pharmaceutical kit of claim 35, comprising 21 to 119 daily doses of estrogen and 21 to 119 daily doses of progestin.

40. The pharmaceutical kit of claim 35, comprising 21 to 91 daily doses of estrogen and 21 to 91 daily doses of progestin.
